(19) United States
(12) Patent Application Publication
(10) Pub. No.: US 2004/0043064 A1
(43) Pub. Date: Mar. 4, 2004

(54) Dosage forms having reduced moisture transmission

(76) Inventors: Theodore L. Iorio, Millis, MA (US); Shubha Chungi, Sharon, MA (US)

Correspondence Address: REED & EBERLE LLP, 800 MENLO AVENUE, SUITE 210, MENLO PARK, CA, 94025, US

(21) Appl. No.: 10/231,677

(22) Filed: Aug. 29, 2002

(51) Int. Cl. A61K 9/48

(52) U.S. Cl. 424/452

(57) ABSTRACT

A pharmaceutical dosage form is provided. The dosage form is comprised of a capsule having a substantially moisture-impermeable outer surface, an enclosed interior cavity, and at least one active agent contained therein. The dosage form exhibits reduced moisture transmission. In addition, methods for reducing moisture transmission in a dosage form and methods of treating a patient are described.

### TECHNICAL FIELD

This invention relates generally to pharmaceutical dosage forms, and more particularly relates to pharmaceutical capsules having reduced moisture transmission. In addition, the invention relates to methods for reducing moisture transmission in pharmaceutical capsules, methods for using the capsules in drug delivery, and methods for treating a patient.

### BACKGROUND

Pharmaceutical scientists routinely encounter formulation challenges resulting from the transmission of moisture into and out of dosage forms. For example, moisture in the atmosphere can degrade a drug by hydrolysis, thereby reducing its shelf life. Moreover, humid conditions represent an ideal environment for the growth of microorganisms with the consequence that the addition of antimicrobial agents and other protective steps are often required in order to prepare a drug formulation for storage. Furthermore, moisture from the atmosphere may cause certain powders, e.g., powders used in pulmonary administration, to agglomerate and form relatively large granules. As powders used in pulmonary administration require a specific particle size range for maximum effectiveness, the phenomenon of granulation in a moist environment represents yet another obstacle that must be addressed. Exacerbating these challenges are the problems associated with improper storage of medicines, e.g., in the bathroom medicine cabinet where moisture from showers or baths creates a humid environment. Conversely, overly dry conditions may cause certain dosage forms to release moisture, thereby making the dosage forms brittle. Consequently, it is clear that moisture transmission in a dosage form often represents a challenge to those in the pharmaceutical arts.

Capsules and the formulations contained therein are particularly susceptible to the often-destructive effects of moisture transmission. For example, capsules made from gelatin, a widely used capsule material, are particularly prone to moisture problems. Gelatin capsules stored under very humid conditions tend to absorb moisture from the air, with the resulting capsules becoming soft, discolored, deformed, and/or otherwise unusable or unappealing to a patient. Perhaps even more importantly, some of the moisture absorbed by the gelatin capsule walls is transmitted to the capsule interior, resulting in any number of problems, including degradation, microbial growth, and agglomeration, as detailed above.

When stored under very dry conditions, gelatin capsules often allow for the transmission of moisture from the capsule interior and capsule walls to the external environment. Under such conditions, the capsules generally become brittle and therefore unsuitable for packaging, shipping, and storage. Furthermore, a brittle capsule is generally unsuitable for handling as the capsule may not be sufficiently resilient to withstand the forces encountered during packaging and shipping.

Some have suggested the use of non-gelatin capsule materials to solve these and other problems. For example, U.S. Pat. No. 4,917,885 to Chiba et al. describes the use of a cellulose-based ether, such as hydroxypropyl methylcellulose, as a capsule material. Although some improvement has been observed with these capsules, they still allow for the permeation of moisture. Thus, dosage forms fabricated from such cellulosic polymers are less than optimal since moisture transmission is often not sufficiently reduced.

Accordingly, there is a need in the art to provide capsules that exhibit reduced moisture transmission. The present invention addresses both this and other needs in the art by providing a capsule having a substantially moisture-impermeable outer surface.

### SUMMARY OF THE INVENTION

Accordingly, it is a primary object of the invention to provide a pharmaceutical dosage form comprising a substantially moisture-impermeable outer surface, an enclosed interior cavity, and at least one active agent contained therein.

It is another object of the invention to provide such a capsule wherein the substantially moisture-impermeable outer surface is comprised of a substantially moisture-impermeable material such as a synthetic polymer.

It is still another object of the invention to provide such a dosage form wherein the capsule is made from a capsule-forming material comprising a synthetic polymer that provides a substantially moisture-impermeable outer surface.

Another object of the invention is to provide such a dosage form wherein the synthetic polymer forms an outer coating over the capsule.

Still another object of the invention is to provide such a dosage form wherein a pharmaceutical formulation is contained within the capsule.

It is an additional object of the invention to provide such a dosage form wherein the pharmaceutical formulation is a dry powder formulation.

It is yet another object of the invention to provide a method for reducing moisture transmission by providing a dosage form as described herein.

It is still another object of the invention to provide a method for treating a patient.

Additional objects, advantages, and novel features of the invention will be set forth in part in the description that follows, and in part will become apparent to those skilled in the art upon examination of the following, and may be learned by practice of the invention.

In one embodiment, a pharmaceutical formulation is provided, comprising a capsule having a substantially moisture-impermeable outer surface, an enclosed interior cavity, and at least one active agent contained therein. The substantially moisture-impermeable outer surface may be effected by any means such as, for example, by laminating a preformed capsule with a polymer or by forming a capsule from a capsule-forming material incorporating a polymer such that the polymer renders the outer surface of the capsule substantially moisture impermeable. As will be appreciated by one of ordinary skill in the art, the outer surface is the coating surface when a substantially moisture-impermeable coating is applied or the outer surface is the capsule wall itself when a capsule is formed from a material incorporating a polymer providing substantial moisture impermeability. Preferred polymers for effecting substantially moisture-impermeable outer surfaces include synthetic polymers, although polymers derived from natural sources, e.g., rubber (i.e., 1,4 cis-polyisoprene) derived from Hevea brasiliensis, may also be used.

Suitable synthetic polymers include, by way of illustration only and not limitation, water-insoluble polymers and even relatively water-soluble polymers, which may be used in certain contexts as will be described in detail below. Exemplary water-insoluble polymers include polyesters, poly(nonhalogenated hydrocarbons), poly(halogenated hydrocarbons), poly(halogenated polyethers), polymers formed from dienes, poly(higher alkylene oxides), polyamides, polysilicones, and poly(acrylonitriles), and combinations thereof. Preferred polymers that have some degree of water-solubility include acrylate polymers, including polyacrylic acids, as well as poly(lower alkylene oxides). Combinations of polymers are included in the present invention, e.g., a combination of two or more water-insoluble polymers or a combination of a relatively water-soluble polymer and a water-insoluble polymer. In addition, alternating copolymers, block copolymers, random copolymers, graft copolymers, terpolymers, block terpolymers, and random terpolymers of any given polymer may be used.

Any active agents may be enclosed in the capsule interior, and the invention is not limited in this regard. Preferably, the active agent is incorporated within a pharmaceutical formulation, e.g., a dry powder. Dry powder formulations are commonly used for pulmonary administration, and preferably comprise one or more active agents that may be administered via pulmonary inhalation. Such active agents include conventional small drug molecules (e.g., bronchodilators and corticosteroids), as well as polypeptide drugs (e.g., LHRH, nafarelin, goserelin, and leuprolide), protein drugs (e.g., insulin, interferon, parathyroid hormone, α₁ proteinase inhibitor, and IL-1 receptor), and nucleic acid drugs (e.g., cystic fibrosis transmembrane conductance gene and α₁ antitrypsin gene).

In another embodiment, a method is provided for reducing moisture transmission in a pharmaceutical dosage form, wherein the method comprises providing a capsule comprising an outer surface, an enclosed interior cavity, and at least one active agent contained therein, wherein the outer surface is comprised of a substantially moisture-impermeable synthetic material comprising a synthetic polymer, thereby producing a dosage form having reduced moisture-transmission properties. The method also includes the optional additional step of sterilizing the dosage form.

In another embodiment, a method is provided for reducing moisture transmission in a dosage form, wherein the method comprises formulating at least one active agent into a capsule; and coating the capsule with a substantially moisture impermeable synthetic polymer to produce a dosage form having reduced moisture-transmission properties.

Another embodiment of the invention provides a method of treating a patient comprising providing a pharmaceutical dosage form as described herein and administering the active agent to a patient in need thereof from the pharmaceutical dosage form.

### BRIEF DESCRIPTION OF THE DRAWINGS

FIG. 1 is a cross-sectional side view of a dry powder inhaler that may be used with current dosage forms of the invention for administering an active agent.

FIG. 2 is a side view of the inhaler of FIG. 1, shown inverted, which is the appropriate position for drug delivery.

### DETAILED DESCRIPTION OF THE INVENTION

I. Overview and Definitions

Before describing the present invention in detail, it is to be understood that this invention is not limited to particular polymers or active agents, as such may vary. It is also to be understood that the terminology used herein is for the purpose of describing particular embodiments only, and is not intended to be limiting.

It must be noted that, as used in this specification and the appended claims, the singular forms “a,” “an,” and “the” include plural referents unless the context clearly dictates otherwise. Thus, for example, reference to “an active agent” includes a single active agent as well as a combination of two or more active agents, reference to “a polymer” includes a single polymer as well as combinations of two or more polymers, reference to “a pharmaceutically acceptable carrier” includes a single pharmaceutically acceptable carrier as well as combinations of two or more pharmaceutically acceptable carriers, and the like.

In describing and claiming the present invention, the following terminology will be used in accordance with the definitions set forth below.

The terms “active agent,” “pharmacologically active agent,” and “drug” are used interchangeably herein to refer to a chemical compound that induces a desired pharmacological and/or physiological effect. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent,” or “drug” are used, then, it is to be understood that applicant intends to include the active agent per se as well as pharmaceutically acceptable, pharmacologically active salts, esters, amides, prodrugs, metabolites, analogs, etc.

By “pharmaceutically acceptable carrier” is meant a material or materials that are suitable for drug administration and not biologically or otherwise undesirable, i.e., that may be administered to an individual along with an active agent without causing any undesirable biological effects or interacting in a deleterious manner with any of the other components of the pharmaceutical formulation in which it is contained.

Similarly, a “pharmacologically acceptable” salt, ester, or other derivative of an active agent as provided herein is a salt, ester, or other derivative that is not biologically or otherwise undesirable.

As provided herein, the terms “effective amount” or “therapeutically effective amount” of an agent are intended to mean a nontoxic yet sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective amount” in any individual case may be determined by one of ordinary skill in the art using routine experimentation.

The terms “treating” and “treatment” as used herein to refer to reduction in severity and/or frequency of symptoms, elimination of symptoms and/or underlying cause, prevention of the occurrence of symptoms and/or their underlying cause, and improvement or remediation of damage.

The terms “condition,” “disease,” and “disorder” are used interchangeably herein to refer to a physiological state that can be prevented or treated by administration of a pharmaceutical formulation as described herein.

The term “patient,” as in treatment of “a patient,” refers to a mammalian individual afflicted with or prone to a condition, disease, or disorder as specified herein, and includes both humans and animals.

“Substantially water impermeable” and “substantially moisture impermeable” are used interchangeably to refer to a capsule surface or capsule wall (also referred to as a capsule shell) that effectively prohibits moisture from reaching the enclosed interior. In a preferred embodiment, such impermeable capsule surfaces or walls prohibit substantial water or moisture permeation into the capsule interior when stored at 40° C. at 75% relative humidity for three days. Water or moisture permeation in a material is also characterized according to the material's moisture vapor transmission rate (MVTR). Ideally, a given capsule surface or capsule wall will have an MVTR of from 0 to less than about 3, more preferably from 0 to less than about 1.5, and most preferably from 0 to less than about 1 g-mm/m²-day-atm. Procedures for testing the MVTR for a given system are known to those of ordinary skill in the art and include, for example, procedure E96 (and variations thereof) developed by the American Society for Testing and Materials (ASTM, West Conshohocken, Pa.).

The term “pulmonary” as used herein refers to any part, tissue, or organ that is directly or indirectly involved with gas exchange, i.e., O₂/CO₂ exchange, within a patient. “Pulmonary” contemplates both the upper and lower airway passages and includes, for example, the mouth, nose, pharynx, oropharynx, laryngopharynx, larynx, trachea, carina, lungs, bronchi, bronchioles, and alveoli. Thus, the phrase “pulmonary drug administration” refers to administering the formulation described herein to any part, tissue, or organ that is directly or indirectly involved with gas exchange within a patient.

“Optional” or “optionally” means that the subsequently described event or circumstance may or may not occur, and that the description includes instances where said event or circumstance occurs as well as instances where it does not. For example, the reference to an “optional pharmaceutically acceptable carrier” in a formulation means that such a carrier may or may not be present, and the description includes formulations wherein a carrier is present and formulations wherein a carrier is not present.

II. The Pharmaceutical Dosage Form

The pharmaceutical dosage form is comprised of a capsule having a substantially moisture-impermeable outer surface, an enclosed interior cavity, and at least one active agent contained therein.

As stated previously, the substantially moisture-impermeable outer surface may be effected by any means. For example, commercially available capsules can be coated with a suitable polymer, thereby rendering the outer surface substantially impermeable to water. In addition, a suitable polymer may be added to a capsule-forming material whereupon the outer surface of the capsule is rendered substantially moisture impermeable. As will be appreciated by one of ordinary skill in the art, the “outer surface” of the capsule is the coated surface when a polymer is coated onto a capsule or the capsule wall itself when the capsule is formed using a capsule-forming material comprising a suitable polymer. In either of these cases, however, the quality of substantial moisture impermeability is effected with a suitable polymer as described herein.

Suitable polymers for use within the present invention include synthetic polymers and natural polymers. Synthetic polymers are preferred since these polymers typically exhibit more desirable characteristics, e.g., greater purity. Polymers derived from natural sources may also be used provided that they are sufficiently processed, e.g., purified. A preferred natural polymer is ethyl cellulose. In addition, the synthetic polymer is preferably non-hydrolyzable. Non-hydrolyzable materials are those that do not undergo substantial degradation in the presence of water. Non-hydrolyzable materials are particularly preferred when the capsule is not intended for systemic, e.g., oral, administration.

Due to their immiscibility in water, water-insoluble polymers are preferred polymers for rendering the outer surface substantially impermeable to water. Examples of water-insoluble polymers include, without limitation: polyesters; poly(nonhalogenated hydrocarbons); poly(halogenated hydrocarbons); poly(halogenated polyethers); polymers formed from dienes; poly(higher alkylene oxides), i.e., poly(C₃₋₁₀ alkyleneoxides); polyamides; polysiloxanes and polysilanes; and poly(acrylonitriles).

Polyester polymers include those polymers having hydrocarbon backbones containing ester linkages. Preferred polyester polymers include: poly(alkylene terephthalates), such as poly(ethylene terephthalate), poly(butylene terephthalate), and poly(trimethylene terephthalate); and poly(alkene naphthalates), such as poly(ethylene naphthalate).

Nonhalogenated hydrocarbon polymers include those polymers formed exclusively from hydrogen and carbon, wherein each monomeric unit preferably has from about 1-20 carbon atoms. Particularly preferred nonhalogenated hydrocarbon polymers include polyethylene, polypropylene, polystyrene, polyisobutylene, and polymethylpentene. Polyethylene includes both high-density polyethylene and low-density polyethylene. By convention, high-density polyethylene has a weight-average molecular weight of greater than 200,000, whereas low-density polyethylene has a weight-average molecular weight of less than 200,000. As will be appreciated by those skilled in the art, a weight-average molecular weight represents the average molecular weight of all the individual molecules in a given sample.

Halogenated hydrocarbon polymers include those hydrocarbon polymers wherein one or more of the hydrogen atoms are substituted with one or more of the same or different halogen atoms (e.g., fluorine, chlorine, bromine, and iodine). These polymers include, without limitation: halogenated ethylenes, including fluorinated ethylenes such as poly(tetrafluoro ethylene) and poly(chlorotrifluoro ethylene); poly(vinyl halides), such as poly(vinyl chloride), poly(vinyl fluoride), poly(vinyl bromide), and poly(vinyl iodide); and poly(vinylidene halides), such as poly(vinylidene chloride), poly(vinylidene fluoride), poly(vinylidene bromide), and poly(vinylidene iodide). Particularly preferred halogenated hydrocarbon polymers are poly(vinyl chloride), poly(vinyl fluoride), poly(vinylidene chloride), and poly(vinylidene fluoride).

Halogenated polyether polymers include those polymers containing ether moieties wherein one or more of the hydrogen atoms are substituted with one or more of the same or different halogen atoms. A preferred halogenated polyether includes chlorinated polyether.

Diene polymers are polymers made from individual monomeric units containing two carbon-carbon double bonds. Following polymerization, however, only one carbon-carbon double bond remains in each monomer. Diene polymers include, without limitation, polybutadiene and poly(dicyclopentadiene).

Higher alkylene oxide polymers are made from C₃₋₁₀ alkylene moieties linked together by ether linkages. Examples of poly(higher alkylene oxides) include, without limitation, poly(butylene oxide), poly(propylene oxide) and poly(phenylene oxides) such as poly(2,6-dimethylphenylene oxide).

Polyamides link individual carbon-containing monomer units via an amide bond. Preferred polyamides are the nylons, which typically comprise four to eighteen carbon atoms between each amide bond. The carbon-containing monomer is often a straight chain of carbon atoms. Nylons are conventionally named based on the number of carbon atoms in each monomer. For example, nylon 6 has six carbon atoms between each nitrogen atom of the amide bond polymer, while nylon 12 has twelve carbon atoms between each nitrogen atom of the amide bond in the polymer.

Polysiloxanes and polysilanes are considered inorganic polymers inasmuch as carbon is not found in the backbone chain. The polymeric backbone of polysiloxanes comprises a chain of alternating silicon and oxygen atoms while polysilanes simply comprise a chain of silicone atoms. In both cases, the silicone atom may have one or two alkyl or other functional groups attached to it. Preferred polysiloxanes include, without limitation, poly(dimethyl siloxane), poly(methylphenyl siloxane) and poly(diphenyl siloxane). Preferred polysilanes include, without limitation, poly(methylphenyl silane).

Poly(acrylonitrile) is a polymer wherein the monomer is represented by —CH₂—CH(CN)—. Although poly(acrylonitrile) can be used according to the present invention, it is preferred that co- or terpolymers containing the acrylonitrile monomer are used. Polymeric arrangements such as those found in copolymers, terpolymers and others are discussed in more detail below.

Preferred relatively water-soluble polymers include, without limitation, poly(lower alkylene oxides) and acrylate polymers, including polyacrylic acids.

Lower alkylene oxide polymers have a repeating C₁₋₂ alkylene moiety linked through ether bonds. Examples of poly(lower alkylene oxides) include, without limitation, poly(ethylene oxide).

Acrylate polymers include those polymers formed from acrylic acid and analogs thereof. Thus, acrylate polymers include, for example: polyacrylic acid per se; poly(methyl acrylate) and other polymers of alkyl esters of acrylic acid, such as poly(ethyl acrylate); polymethacrylic acid and other polymers of alkylated acrylic acids, such as polyethylacrylic acid; and poly(methyl methacrylate) and other polymers of alkylated alkyl esters of acrylic acid, such as poly(ethyl methacrylate). Preferably, the alkyl esters of acrylic acids and methacrylic acids have from about 1-8 carbon atoms (straight or branched) in the alkyl group. A particularly preferred acrylate polymer is poly(methyl methacrylate).

Combinations of polymers may be used and are particularly preferred when relatively water-soluble polymers are used, e.g., a combination of a relatively water-soluble polymer and a water-insoluble polymer. In addition, heteropolymers or polymers comprising two or more different monomeric units may be used. Examples of heteropolymers include alternating copolymers, block copolymers, random copolymers, graft copolymers, terpolymers, block terpolymers, and random terpolymers. The monomeric units comprising the heteropolymer are preferably, although not necessarily, selected from those provided in the polymers described herein. Preferred copolymers include poly(acrylonitrile-co-vinyl chloride). Preferred terpolymers include poly(acrylonitrile-co-butadiene-co-styrene).

The synthetic polymer used herein may be used alone or mixed with one or more other polymers. Furthermore, homopolymers, alternating copolymers, block copolymers, random copolymers, graft copolymers, terpolymers, block terpolymers, and random terpolymers comprising any of the monomeric units provided in the polymers described herein may be used.

Although one or any combination of any of the foregoing polymers is preferred, the polymer used in the capsule or coating must render the capsule substantially moisture impermeable. For example, when the polymer provides a relatively high amount of moisture impermeability, a relatively thin capsule wall and/or coating is generally used since a relatively small amount of the polymer necessary. When the polymer provides a relatively low amount of moisture impermeability, a relatively thick capsule wall and/or coating is required since a relatively large amount of the polymer is necessary to render the dosage form substantially water impermeable.

The polymer is chosen based on many factors, such as its ability to prevent or reduce the transmission of moisture, ease with which the polymer can be included in a capsule-forming material and/or coating, and so forth. Although there are many ways to characterize the property of a given polymer to prevent or reduce the transmission of moisture, a conventional characterization is based the polymer's moisture vapor transmission rate (MVTR). The MVTR of any particular polymer can be determined using standardized tests known to those of ordinary skill in the art. Such standardized tests include, for example, American Society for Testing and Materials (ASTM, West Conshohocken, Pa.) procedure E96 or a variation thereof. This procedure, as well as others, standardizes temperature, atmospheric pressure, moisture content, and thickness of the polymeric film. Representative MVTR values for certain synthetic polymers are provided in Table 1 below:

TABLE 1
Representative MVTR Values for Certain Synthetic Polymers

| Synthetic Polymer | MVTR (g-mm/m²-day-atm) |
|---|---|
| Polyesters | 0.58 |
| Polypropylene | 0.17 |
| Polyethylene (low density) | 0.41 |
| Polyethylene (high density) | 0.16 |
| Poly (vinyl chloride) | 1.20 |
| Polyamide (nylon) 6* | 1.00 |
| Polyamide (nylon) 12* | 0.40 |

*numeral refers to the number of carbon atoms in each monomer.

MVTR and related moisture permeation information for polymers are provided in the relevant literature and in, for example, the Polymer Handbook, Third Edition (New York: John Wiley & Sons, Inc., 1989) and Remington: The Science and Practice of Pharmacy, Twentieth Edition (Easton, Pa.: Mack Publishing Co., 2000).

Preferably, the polymer inherently provides a high degree of moisture impermeability when present in the capsule coating or capsule wall, as represented by an MVTR value of from 0 to less than about 3, more preferably from 0 to less than about 1.5, and most preferably from 0 to less than about 1 g-mm/m²-day-atm. Consequently, it is preferred that the resulting capsule itself has an MVTR value within these ranges as well. Of course, synthetic polymers having MVTR values outside of these ranges may still be used so long as greater amounts (e.g., to form a thicker capsule coating or capsule wall) of the synthetic polymer are used as described below.

As will be appreciated by one of ordinary skill in the art, the amount of the polymer used and the resulting thickness of the capsule wall or coating can be determined easily by one of ordinary skill in the art using only routine experimentation. For example, a series of powder-containing dosage forms, each including serially increased amounts of a given polymer, can be tested for impermeability to moisture by placing the dosage form in a humid environment. After sufficient exposure to the humid environment, e.g., seven days, the contents of each dosage form are weighed and compared with the corresponding powder weight prior to placement in the humid environment. Those capsules housing powders where the weight does not substantially change after exposure to the humid environment possess a sufficient amount of the polymer necessary to provide substantially moisture impermeable dosage forms. Other procedures for determining the amount of the polymer necessary to render the dosage form impermeable to moisture can be used as well.

Once selected, the polymer is used in the formation of a capsule itself and/or is used in a coating surrounding the capsule. In either case, it is the polymer that renders the capsule substantially impermeable to moisture.

When the polymer is used in the formation of the capsule itself, the polymer is used either alone or in combination with one or more conventional capsule-forming components, e.g., gelatin or hydroxypropyl methylcellulose. For example, gelatin can be mixed with the synthetic polymer or can form a layer beneath the polymer outer surface.

For purposes herein, a capsule-forming material shall refer to the polymer alone or with one more conventional capsule-forming components. Capsules are produced from the capsule-forming material using conventional techniques known to those of ordinary skill in the art and described in, for example, U.S. Pat. No. 4,917,885. Briefly, the polymer or admixture thereof is optionally mixed with one or more excipients, e.g., plasticizer, colorant, etc., and is heated to an appropriate temperature to form a liquid. Thereafter, capsule-shaped pins corresponding to the size and shape of the desired capsule half are dipped into the liquid. The pins are then slowly removed from the liquid and cooled. Once cooled, the material on the pin forms a solid film representing a capsule half. The capsule half is then removed from the pin by mechanical or manual means. Alternatively, the capsule may be prepared using conventional extrusion molding techniques.

Plasticizers may be added to the capsule-forming material or the coating mixture to reduce the brittleness of the capsule shells. Typical plasticizers include, for example, glycerin, propylene glycol, polyethylene glycol, dibutyl sebacate, diethylphthalate, triethyl citrate, and combinations thereof. Typically, the plasticizer, when present, represents from about 5-40 wt %, more preferably from about 10-30 wt %, of the capsule-forming material or coating mixture.

The capsule-shaped pins may be formed such that the capsule made therefrom has additional features. For example, the pin may include one or more circular grooves on the outer circumference of the pin, thereby providing a means to positively join or “lock” two capsule halves together. This feature is included on CONI-SNAP™ and CONI-SNAP SUPRO™ capsules, which are available from Capsugel, Greenwood, S.C. In addition, the capsule-shaped pins may be tapered. The capsule-shaped pins may also be formed so as to introduce other features into the corresponding capsule.

The capsule halves are sized such that a capsule having the desired dimension is formed upon proper joining of each capsule half. The capsules described herein are sized to hold the desired amount of powdered material, typically up to about 1,000 mg of a formulation. Preferably, although not necessarily, the size of any particular capsule described herein will correspond to a conventional capsule size, e.g., to a size on the conventional capsule scale of, from largest to smallest, 000, 00, 0, 1, 2, 3, 4, or 5. See, for example, Remington: The Science and Practice of Pharmacy, Twentieth Edition, supra, for additional information concerning these and other conventional capsule sizes. Although the maximum weight that may be housed in the capsule will depend upon the density of the formulation, generally a size 000 capsule can house about 600 mg of formulation while a size 5 capsule can house about 30 mg. Of course, as will be explained in more detail below, the entire volume of the capsule need not be occupied with the formulation and/or active agent.

Once the capsule halves are formed, an active agent or formulation containing an active agent is metered out and placed into a first capsule half. The active agent or formulation thereof is typically in powdered form. A corresponding mating capsule half is then nested or fitted to the first capsule half, thereby forming a complete capsule having an interior volume. The amount of the active agent or formulation thereof is metered out and subsequently housed within the capsule interior, and may represent only a small fraction of the available interior volume. Such small fill volumes are particularly preferred when the capsule is used in conjunction with pulmonary administration of an active agent. Preferred fill volumes in this case are typically less than about 50%, preferably less than about 25%, with fill volumes of less than about 15% being most preferred. The active agent or formulation containing an active agent minimally occupies about 0.01% of the available fill volume. In other applications, such as those intended for packaging powders used for oral administration, the fill volume may represent as much as from about 90% up to 100% of the available fill volume.

Typically, although not necessarily, a capsule-filling machine is used to meter and place the active agent or active agent-containing formulation into the capsule. Such machines are commercially available from, for example, Capsugel, Greenwood, S.C. and M.O. Industries, Whippany, N.J. In addition, manual processes may be used to add an active agent or an active agent-containing formulation into the capsule. One such method includes placing the formulation on a clean and nonporous surface and compressing the formulation using a spatula. One capsule half is “punched” into the compacted formulation such that a portion of formulation is retained in the capsule half. The “punching” action may be repeated in other areas of the compacted formulation until the desired amount of the formulation is placed in the capsule half. Regardless of which filling method is used, the fill amount may be verified by testing the capsules, e.g., by weighing the capsules (deducting for the weight of an unfilled capsule) or emptying the contents of representative capsules and measuring the amounts contained therein by analytical, e.g., chromatographic, methods. The fill method can thereafter be modified, e.g., to include either more or less of the formulation, if it is found that the desired amount of the active agent or formulation thereof is not contained in the capsule.

Once the capsule halves are joined, the capsule may optionally be sealed. Any capsule-sealing technique known to those skilled in the art may be used. For example, a gelatin band may be placed around the capsule and then heated, thereby sealing the capsule. Other capsule-sealing techniques may also be used. Advantageously, sealed capsules show evidence of tampering; they also thwart efforts of individuals attempting to tamper with the capsule contents. In addition, sealed capsules safeguard against contaminants entering the capsule interior.

Alternatively, or in addition to including the polymer in the capsule itself, the polymer used to render the capsule substantially impermeable to water can be coated onto the capsule. Consequently, conventional capsules are rendered substantially impermeable to moisture in this way. Coating a capsule with a suitable polymer as described herein results in a capsule having a substantially moisture-impermeable outer surface wherein the capsule itself represents an inner layer of material relative to the coating. The capsule may be coated with the polymer using conventional coating techniques known to those skilled in the pharmaceutical arts. For example, the capsule may be coated employing an air suspension coating process. In such a process, the capsules in which a formulation has been previously added (see discussion supra for techniques for adding formulations to capsules), are placed in a chamber and suspended in air via a blower located at the bottom of the cylinder. As the polymer is introduced into the bottom of the cylinder, the polymer is also suspended in the air and allowed to contact the capsules, thereby coating the capsule. Other techniques may be used, including coating the capsule halves before adding the formulation and coating a capsule formed with a polymer as described herein so as to increase the capsule's relative impermeability to moisture.

The capsules may also undergo a sterilization treatment. Methods for sterilizing dosage forms are known and include exposure of the dosage form to a sterilizing medium, e.g., radiation (including gamma radiation and beta radiation) or a sterilizing gas such as ethylene oxide. Briefly, the dosage form is placed in a sterilization chamber and exposed to the sterilizing medium for an amount of time effective to sterilize the dosage form. Suitable sterilization times are known to those skilled in the art or can be determined through routine experimentation. When ethylene oxide is used, the chamber and dosage form are purged of any remaining ethylene oxide by exposure to a pressurized gas, e.g., carbon dioxide.

The capsules may be filled with any material and are not limited in this regard. Preferably, the capsules are filled with a pharmaceutical formulation, which comprises the active agent, alone or in combination with one or more pharmaceutical excipients. The capsules are not limited with respect to the active agent so long as the active agent is suitable for storage in a capsule. The capsules described herein, however, preferably contain an active agent and/or formulation for which protection from water is desired. For example, the capsules described herein are suited to house an active agent for which hydrolysis poses a problem. In addition, the capsules are suited to house a formulation, e.g., a dry powder formulation used in pulmonary administration, subject to moisture-induced granulation.

The active agent, either alone or as part of a dry powder formulation, can be any active agent suitable for pulmonary administration. Such active agents are known or can be determined by one of ordinary skill in the art. The active agent can be intended for systemic administration or the active agent can be intended to produce localized effects in the lungs. The active agent can be a narcotic, e.g., an opiate derivative such as morphine, a bronchodilator, a corticosteroid, an anti-cystic fibrosis agent, an anti-diabetes agent, an immunoregulatory agent, a hormone, a hormone modulator, and so forth. The active agent is preferably a small drug molecule, a polypeptide drug, a protein drug, a polynucleotide drug, or a combination thereof.

Preferred small molecule drugs for use in the present invention include, without limitation, those selected from the group consisting of bronchodilators, corticosteroids, and combinations thereof. Bronchodilators from the pharmacological classes of β₂ adrenergic agonists, anticholinergics, and/or xanthine derivatives may be incorporated into the formulations. It is preferred, however, that the bronchodilator has agonist activity for β₂ adrenergic receptors. Furthermore, the formulation is not limited to one bronchodilator, as combinations of bronchodilators may also be present.

Particularly preferred among the β₂ adrenergic agonists are albuterol, bitolterol, clenbuterol, fenoterol, formoterol, levalbuterol (i.e., homochiral (R)-albuterol), metaproterenol, pirbuterol, procaterol, reproterol, rimiterol, salmeterol, terbutaline, pharmacologically acceptable salts, esters, isomers, active metabolites, prodrugs, and other derivatives thereof, and combinations of any of the foregoing. Most preferred, however, are pirbuterol acetate, pirbuterol dihydrochloride, levalbuterol sulfate, and levalbuterol hydrochloride.

Particularly preferred corticosteroids include those selected from the group consisting of beclomethasone, betamethasone, budesonide, dexamethasone, flunisolide, hydrocortisone, triamcinolone, fludrocortisone, fluocinolone, fluocinonide, fluticasone, methylprednisolone, mometasone, prednisone, and pharmacologically acceptable salts, esters, isomers, active metabolites, prodrugs, and other derivatives thereof. Preferred pharmacologically acceptable esters include those esters derived from C₂₋₆ carboxylic acids. More specifically, preferred esters are derived from corresponding carboxylic acids having two to six carbon atoms that are branched, unbranched, or cyclic, saturated or unsaturated, aromatic or nonaromatic, and heteroatom substituted or unsubstituted. It is particularly preferred for mometasone, however, that the ester derivative is mometasone furoate, mometasone thiophene ester, or mometasone monoacetate, with mometasone furoate most preferred. Furthermore, it is preferred that anhydrous mometasone furoate is present in the formulation, although hydrate derivatives, e.g., mometasone furoate monohydrate, may also be used.

Any polypeptide, protein or polynucleotide drug suitable for pulmonary inhalation may be housed in the capsules described herein. Such drugs are known or can be identified by those of ordinary skill in the art. Preferred polypeptide drugs include, without limitation, luteinizing hormone-releasing hormone (LHRH), nafarelin, goserelin, leuprolide, and cyclosporin. Preferred protein drugs include, without limitation, calcitonin, granulocyte colony-stimulating factor, growth hormones, heparin, parathyroid hormone, insulin, interferon, α₁-proteinase inhibitor, and interleukins (e.g., IL-1). Preferred polynucleotide drugs include, without limitation, cystic fibrosis transmembrane conductance (CFTR) gene and α₁-antitrypsin gene. For polynucleotide drugs, certain excipients may be used to facilitate delivery of the agent to the host. Examples of such excipients include DOPE (dioleoylphosphatidyl-ethanolamine), DOTAP (N-(1-(2,3-dioleoyloxy)propyl)-N,N,N-trimethylammoniumethyl sulphate), DOTMA (N-(1-(2,3-dioleoyloxy)propyl)-N,N,N-trimethylammonium chloride), DEAE-dextran (diethylaminoethyl-dextran), albumin, cellulose, collagens, dextrins, glycine, and sugars (e.g., glucose, lactose and ribose).

The active agent or each active agent housed in the capsule, whether identified above or not, may be administered in the form of a pharmacologically acceptable salt, ester, amide, prodrug, derivative, stereoisomer, or as a combination thereof. Salts, esters and derivatives of the active agents may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, acid addition salts are prepared from the free base (e.g., compounds having a neutral —NH₂ or cyclic amine group) using conventional means, involving reaction with a suitable acid. Typically, the base form of an active agent is dissolved in a polar organic solvent such as methanol or ethanol and the acid is added at a temperature of about 0-100° C., preferably at ambient temperature. The resulting salt either precipitates or may be brought out of solution by addition of a less polar solvent. Suitable acids for preparing the acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like as well as inorganic acids, e.g., hydrochloric acid, hydrobromic acid, sulfuric acid, nitric acid, phosphoric acid, and the like. An acid addition salt may be reconverted into the free base by treatment with a suitable base. Preparation of basic addition salts of an active agent having an acid moiety (e.g., carboxylic acid group or hydroxyl group) are prepared in a similar manner using a pharmaceutically acceptable base. Suitable bases include both inorganic bases, e.g., sodium hydroxide, potassium hydroxide, ammonium hydroxide, calcium hydroxide, magnesium hydroxide, and the like, as well as organic bases such as trimethylamine, and the like. Preparation of esters involves functionalization of hydroxyl and/or carboxyl groups that may be present within the molecular structure of the drug. The esters are typically acyl-substituted derivatives of free alcohol groups, i.e., moieties that are derived from carboxylic acids of the formula RCOOH where R is alkyl, and preferably is lower, i.e., C₁₋₆ alkyl. Esters can be reconverted to the free acids, if desired, by using conventional hydrogenolysis or hydrolysis procedures. Preparation of amides and prodrugs can be carried out in an analogous manner. Other derivatives of the active agents may be prepared using standard techniques known to those skilled in the art of synthetic organic chemistry, or may be deduced by reference to the pertinent literature and texts.

Stereoisomers in substantially pure form may also be used. Individual stereoisomers may have unique or beneficial properties that make that individual isomer particularly well suited as an active agent. Consequently, individual stereoisomers or mixtures thereof of the active agents are included. Thus, the active agent may be present in the formulation as a racemate, i.e., equal amounts of a stereoisomer, a pure form, e.g., levalbuterol, or a mixture of nonequal amounts of two stereoisomers, e.g., nonequal amounts of (S)-albuterol/(R)-albuterol, and so on.

The various hydrates of the active agent and other formulation components, if present, may be used. As is known, one or more water molecules may associate with a particular compound based on, for example, the availability of hydrogen bonding. Methods of producing hydrated species are known and include, for example, placing the active agent in a humid environment. In addition, methods of removing one or more water molecules are known and include, by way of example, exposing the active agent to dry heat.

As noted above, the formulation of the present invention may also contain various excipients, provided such excipients do not have a deleterious effect on the intended patient or have a deleterious chemical or physical effect on any component in the formulation. Thus, for example, excipients such as preservatives, surface active agents, buffering agents, suspending agents, and the like can be combined with the formulation. The type and amount of any excipient will depend on the type of formulation, the device used for administration, and the intended route of administration, as will be appreciated by one of ordinary skill in the art. Specific examples of each of these excipients are well known by those skilled in the art of pharmaceutical formulation.

For dry powder formulations intended for pulmonary administration, the formulation may also include one or more pharmaceutically acceptable carriers. Although any carrier suitable for pulmonary drug administration may be used, pharmaceutical sugars are particularly preferred for use as carriers. Preferred pharmaceutical sugars include those selected from the group consisting of fructose, galactose, glucose, lactitol, lactose, maltitol, maltose, mannitol, melezitose, myoinositol, palatinite, raffinose, stachyose, sucrose, trehalose, xylitol, hydrates thereof and combinations thereof. It is particularly preferred, however, that lactose serves as the carrier. Often, the carriers are obtained commercially, e.g., PHARMATOSE™ 325 brand of lactose monohydrate available from DMV International, Veghel, The Netherlands.

In addition, for dry powder formulations intended for pulmonary administration, the active agent(s) preferably has a particle size diameter of between about 0.1-65 μm. It is most preferred that the active agent particles be about 1-10 μm, more preferably about 2-5 μm in diameter. The remaining components, particularly the carrier will typically, have a particle size from about 30-100 μm in diameter, with sizes from about 30-70 μm being preferred.

For any given particle size range, it is preferred that at least about 60%, more preferably at least about 70%, still more preferably at least about 85%, of the stated particles have a size within the stated or given range. It is most preferred, however that at least about 90% of the particles have the size in the stated or given range. For example, when a component is stated to have a particle size less than 10 μm, it is most preferred that at least 90% of the particles of that component have a particle size of less than 10 μm.

III. Utility and Administration

The capsules described herein are solid dosage forms that protect the enclosed pharmaceutical formulation from the deleterious effects of moisture. In addition, the invention also relates to methods for reducing moisture transmission in a dosage form. Typically, at least one active agent is formulated into a capsule, and the capsule is coated with a substantially moisture impermeable synthetic polymer to produce a dosage form having reduced moisture-transmission properties

There are several advantages to the dosage forms of the invention. For example, the substantially moisture-impermeable capsule reduces hydrolysis of any susceptible active agent contained therein. In addition, the capsule minimizes or eliminates entirely moisture-induced particle aggregation. Such an advantage is particularly useful for those embodiments where the particle size of the formulation is critical, e.g., in powders for inhalation or sustained-release formulations.

The capsules are used to provide a substantially moisture-impermeable “housing” for a moisture-sensitive active agent or formulation. Capsules not intended for direct administration can include polymers that are toxic or otherwise unsafe for human consumption. In these cases, the capsule may house any active agent or formulation that would benefit from protection from moisture. Furthermore, tablets, other dosage forms, or formulations may be contained in the capsule, thereby protecting the dosage form from moist or humid environments. In this case, the dosage form or formulation is removed from the capsule and immediately administered. In this respect, the capsule will preferably contain a unit dose of the active agent. Thus, for example, if the capsule contains a powder formulation, a unit dose of the active agent is preferably contained in the capsule, thereby eliminating the need for the patient to measure out the dose.

It will be noted that the capsule contents may be administered in any appropriate manner. For example, pulmonary inhalation may be used to administer a suitably formulated powder as discussed above. Although other methods for administering powders may be used, pulmonary administration of powders contained in the capsules is generally provided by one of two methods.

In one method, the capsule provides a unit dose of the formulation. The capsule is manually separated or otherwise opened and the powder formulation contained in the capsule is inhaled, generally with the aid of a powder inhaler. In this method, the capsule does not play a direct role in the administration of the formulation, but serves as a substantially moisture impermeable housing providing a premeasured dose of the formulation.

In another method, the capsule plays more of a direct role in the administration of the formulation. For example, some dry powder inhalers include a cutting means, e.g., a knife, which creates one or more slits or openings in the capsule. These openings provide the means for the powder formulation to exit the capsule. In some circumstances the formulation becomes airborne within the capsule, forming what has been described as a “dancing cloud” phenomenon. The airborne particles are then easily inhaled, thereby effecting pulmonary administration. Advantageously, the capsules described herein slit cleanly, thereby minimizing the formation of debris or dust that might be inhaled by a patient.

The invention also includes a method of treating a patient comprising first providing a pharmaceutical dosage form comprised of a capsule having a substantially moisture-impermeable outer surface, an enclosed interior cavity, and an active agent followed by administering a therapeutically effective amount of the active agent to a patient in need thereof from the pharmaceutical dosage form. Preferably, a patient inhales the active agent contained in the capsule, optimally through a dry powder inhaler.

The amount of the active agent administered will, of course, be dependent on the active agent being administered, the subject being treated, the subject's weight, and the judgment of the prescribing physician. The amount of the active agent administered in any particular case, however, can be determined by one of ordinary skill in the art based upon routine experimentation and/or reference to the relevant texts and literature.

Preferably, the active agent is administered in an amount of from about 1 μg/kg to about 100 mg/kg (amount of drug per kilogram body weight of the patient), more preferably from about 10 μg/kg to about 20 mg/kg. Depending on the patient's response, additional dosages within these ranges may be administered. The formulation housed in the capsule is generally, although not necessarily in solid form, e.g., a powder, granulate, tablet, and so forth. In addition, the formulation may be in the form of liquid as in, for example, a liquid-filled capsule. Furthermore, the formulation may be semi-solid, e.g., and housed in the dosage form. The invention, however, is limited neither with respect to the amount of active agent in the formulation nor the particular state, e.g., liquid, solid, and semi-solid, of the formulation.

When a bronchodilator and/or corticosteroid is present in the dosage form, the method is particularly useful in treating a patient suffering from asthma, exercise-induced asthma, bronchitis, bronchospasm, rhinitis, or emphysema. When the active agent is α₁-proteinase inhibitor, cystic fibrosis transmembrane conductance (CFTR) gene, or α₁-antitrypsin gene, patients suffering from cystic fibrosis will benefit from the present method. Patients suffering from diabetes (all types) benefit from the present method of treatment when the dosage form contains an anti-diabetes agent such as insulin.

IV. Inhalers

Preferably, a dry powder inhaler is used to administer the contents of a capsule described herein that contains a dry powder formulation intended for pulmonary administration of an active agent. Dry powder inhalers are well known to those skilled in the art. Preferably, the dry powder inhaler includes or houses at least one dosage form of the invention containing a unit dose of a dry powder formulation intended for pulmonary administration. The patient self-administers the dose by inhaling (via oral or nasal inhalation) the dry powder formulation in the capsule with the assistance of the inhaler. In this manner, delivery of the dry powder formulation to the pulmonary system is effected.

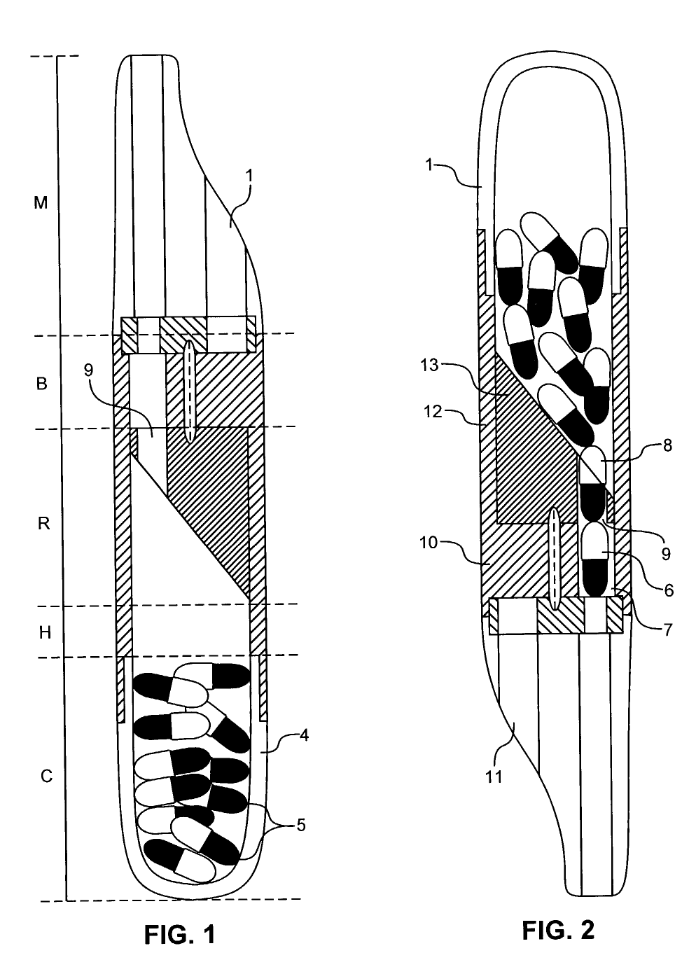

One example of a particularly preferred dry powder inhaler for use with the present capsules is described in U.S. Pat. No. 5,673,686 to Villax et al. and U.S. Pat. No. 5,881,721 to Bunce et al. Specifically, as shown in FIG. 1, a dry powder inhaler 1 comprises a mouthpiece M, a barrel area B, a ramp area R, free headspace H, and a capsule container area C. The capsule container 4 is filled to the brim with capsules 5 as described herein. FIG. 2 shows the same inhaler 1 that has been inverted. The capsules now fill the free headspace and the ramp area and become vertically oriented as they near the passage 9. One capsule 8 is already inserted into the passage 9 and its movement is blocked by the capsule 6 which has preceded it and been dispensed into the capsule chamber 7. The capsule chamber 7 is contained inside a rotating barrel 10.

The operation of the inhaler requires that once a capsule has been loaded into the capsule chamber 7, the rotating barrel 10 be turned. This movement transports the capsule 6 past two small blades (not shown) that slit both ends and carry the capsule to the inhalation position. Once inhalation has taken place, a further turn of the barrel 10 delivers the capsule to the ejection position 11. Continuing to turn the rotating barrel 10 brings the capsule chamber 7 in alignment again with the passage 9 where the next capsule 8 is in place for dispensing.

The rotating barrel 10 is connected to the cylindrical tube 12 and is unconnected to the ramp 13. In operation, the turning motion of the rotating barrel 10 and cylindrical tube 12 is in the opposite direction to that of the ramp 13. These opposite turning motions further assist the righting of the capsules between the ramp 13 and the cylindrical tube 12 and the dispensing of the capsule into the passage 9.

Thus, the dry powder inhaler comprises a tube, a ramp, and a dispensing passage. The tube receives the dosage form of the invention that must be properly oriented. The ramp has a surface that extends substantially across the tube from one wall to an opposite wall. An elongate dispensing passage has a diameter less than that of the tube and is sized to receive the capsule to be dispensed, but only when the axis of the capsule is generally parallel to the axis of the passage. The elongate dispensing passage extends from an inlet end formed by an aperture in the ramp's surface to a dispensing outlet, the passage being adjacent to the one wall of the tube such that the axis of the passage is parallel to, but radially offset from, an axis of the tube. The arrangement is such that when the apparatus is positioned with the passage below the tube and the axis of the passage substantially vertical, a capsule located in the tube will be guided by the ramp surface towards the inlet end of the passage.

Once the capsule has been properly aligned and pierced, the patient inserts the end of the mouthpiece of the inhaler into his or her mouth and inhales. Air enters through the device via any path but generally though specialized air inlets (not shown) on the device. As air enters the inhaler, at least a portion is drawn through an upstream slit. As it travels through the upstream slit into the pierced capsule, the air fluidizes or entrains the powder in the pierced capsule creating what has been referred to as a “dancing cloud.” As suction continues from the patient, the powder-containing air exits through a downstream slit in the pierced capsule and enters the bore of the mouthpiece for passage into the patient's pulmonary system.

Additional dry powder inhalation devices suitable for use with the present capsules include, for example, TURBUHALER® (Astra Pharmaceutical Products, Inc., Westborough, Mass.), ROTAHALER® and DISKHALER® devices (both available from Allen & Hanburys, Ltd., London, England).

It is to be understood that while the invention has been described in conjunction with the preferred specific embodiments thereof, that the foregoing description as well as the examples that follow are intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

### EXPERIMENTAL

The practice of the present invention will employ, unless otherwise indicated, conventional techniques of the pharmaceutical industry and the like, which are within the skill of the art. Such techniques are explained fully in the literature.

In the following examples, efforts have been made to ensure accuracy with respect to numbers used (e.g., amounts, temperatures, etc.) but some experimental error and deviation should be accounted for. Unless indicated otherwise, temperature is in degrees Celsius and pressure is at or near atmospheric at sea level. All reagents were obtained commercially unless otherwise indicated.

### Example 1

Equal amounts of vitamin C beads are placed in size 4 gelatin capsules and sealed. The capsules are separated into two lots: a first lot that receives a coating of high-density polyethylene (heated into liquid form for application to each capsule in the lot); and a second lot of capsules that remain uncoated. All capsules are then maintained in an environment having a temperature of 40° C. and a relative humidity of 75%. After three days, the vitamin C contained in the coated capsules shows no noticeable change while the vitamin C contained in the uncoated capsules exhibits yellowing as a result of moisture permeating into the capsule interior. It is concluded that the capsules coated with high-density polyethylene are substantially impermeable to moisture.

### Example 2

The procedure of Example 1 is repeated except that polypropylene is used in place of high-density polyethylene. The results show that the vitamin C contained in the uncoated capsules exhibits yellowing as a result of moisture permeating into the capsule interior. It is concluded that the capsules coated with polypropylene are substantially impermeable to moisture.

### Example 3

High-density polyethylene capsule halves are prepared. Briefly, the tips of appropriately sized capsule-shaped pins are partially submerged into a liquid formed from heating high-density polyethylene pellets (E. I. du Pont de Nemours and Company, Inc., Wilmington, Del.). The liquid coats the submerged portion of each capsule-shaped pin. The capsule-shaped pins are removed from the high-density polyethylene liquid and allowed to cool. After cooling to room temperature, each capsule half is removed from the capsule-shaped.

Equal amounts of vitamin C beads are placed and sealed in equally sized gelatin (commercially obtained), hydroxypropyl methylcellulose (commercially obtained) and the high-density polyethylene capsules. All capsules are then maintained in an environment having a temperature of 40° C. and a relative humidity of 75%. After three days, the vitamin C contained in the capsules made of high-density polyethylene shows no noticeable change while the vitamin C contained in the gelatin and hydroxypropyl methylcellulose capsules exhibits yellowing of the vitamin C beads as a result of moisture permeating into the capsule interior. It is concluded that the high-density polyethylene capsules are substantially impermeable to moisture.

### Example 4

The procedure of Example 2 is repeated except that the capsule halves are made from a 50:50 mixture of high-density polyethylene and hydroxypropyl methylcellulose. The results show that the vitamin C contained in the capsules made from the mixture of high-density polyethylene and hydroxypropyl methylcellulose does not exhibit yellowing when exposed to a temperature of 40° C. and a relative humidity of 75%. It is concluded that the capsules made from a mixture of high-density polyethylene and hydroxypropyl methylcellulose are substantially impermeable to moisture.

What is claimed is:

1. A pharmaceutical dosage form comprised of a capsule having a substantially moisture-impermeable outer surface, an enclosed interior cavity, and at least one active agent contained therein.

2. The dosage form of claim 1, wherein the substantially moisture-impermeable outer surface is comprised of a substantially moisture-impermeable synthetic polymer.

3. The dosage form of claim 2, wherein the synthetic polymer is non-hydrolyzable.

4. The dosage form of claim 2, wherein the outer surface is comprised of the synthetic polymer.

5. The dosage form of claim 4, wherein the outer surface further comprises hydroxypropyl methylcellulose or gelatin mixed with the synthetic polymer.

6. The dosage form of claim 4, wherein the outer surface further comprises a layer of hydroxypropyl methylcellulose or gelatin beneath the outer surface.

7. The dosage form of claim 2, wherein the synthetic polymer is selected from the group consisting of polyesters, poly(nonhalogenated hydrocarbons), poly(halogenated hydrocarbons), poly(halogenated polyethers), polymers formed from dienes, poly(higher alkylene oxides), polyamides, polysiloxanes, polysilanes, poly(acrylonitriles), poly(lower alkylene oxides), acrylate polymers, polyacrylic acids and alternating copolymers, block copolymers, random copolymers, graft copolymers, terpolymers, block terpolymers, and random terpolymers thereof, and combinations of any of the foregoing.

8. The dosage form of claim 7, wherein the synthetic polymer is selected from the group consisting of poly(ethylene terephthalate), poly(butylene terephthalate), poly(trimethylene terephthalate), poly(ethylene naphthalate), polyethylene, polypropylene, polystyrene, polyisobutylene, polymethylpentene, poly(tetrafluoro ethylene), poly(chlorotrifluoro ethylene), poly(vinyl chloride), poly(vinyl fluoride), poly(vinyl bromide), poly(vinyl iodide), poly(vinylidene chloride), poly(vinylidene fluoride), poly(vinylidene bromide), poly(vinylidene iodide), chlorinated polyether, polybutadiene, poly(dicyclopentadiene), poly(butylene oxide), poly(propylene oxide), poly(2,6-dimethylphenylene oxide), nylons, poly(dimethyl siloxane), poly(methylphenyl siloxane), poly(diphenyl siloxane), poly(methylphenyl silane), poly(acrylonitrile), poly(ethylene oxide), poly(methyl acrylate), poly(ethyl acrylate), polymethacrylic acid, polyethylacrylic acid, poly(methyl methacrylate), poly(ethyl methacrylate), and alternating copolymers, block copolymers, random copolymers, graft copolymers, terpolymers, block terpolymers, and random terpolymers thereof, and combinations of any of the foregoing.

9. The dosage form of claim 8, wherein the synthetic polymer is selected from the group consisting of polyethylene, polypropylene, polystyrene, and combinations of any of the foregoing.

10. The dosage form of claim 1, wherein the active agent is contained in a pharmaceutical formulation.

11. The dosage form of claim 10, wherein the pharmaceutical formulation is a dry powder.

12. The dosage form of claim 11, wherein the dry powder represents no more than 15 vol % of the volume of the cavity.

13. The dosage form of claim 1, wherein the active agent has a particle size diameter within the range of about 0.1-65 μm.

14. The dosage form of claim 13, wherein the active agent has a particle size diameter within the range of about 1-10 μm.

15. The dosage form of claim 1, wherein the active agent is selected from the group consisting of small drug molecules, polypeptides, proteins, and polynucleotides.

16. The dosage form of claim 15, wherein the active agent is selected from the group consisting of narcotics, bronchodilators, corticosteroids, anti-cystic fibrosis agents, anti-diabetes agents, immunoregulatory agents, hormones, and hormone modulators.

17. The dosage form of claim 16, wherein the active agent is selected from the group consisting of albuterol, bitolterol, clenbuterol, fenoterol, formoterol, levalbuterol, metaproterenol, pirbuterol, procaterol, reproterol, rimiterol, salmeterol, terbutaline, beclomethasone, betamethasone, budesonide, dexamethasone, flunisolide, hydrocortisone, triamcinolone, fludrocortisone, fluocinolone, fluocinonide, fluticasone, methylprednisolone, mometasone, prednisone, luteinizing hormone-releasing hormone, nafarelin, goserelin, leuprolide, cyclosporin, calcitonin, granulocyte colony-stimulating factor, growth hormones, heparin, parathyroid hormone, insulin, interferon, α₁-proteinase inhibitor, interleukins, cystic fibrosis transmembrane conductance gene, α₁-antitrypsin gene, and pharmacologically acceptable salts, esters, and derivatives thereof.

18. The dosage form of claim 16, wherein the active agent is a corticosteroid.

19. The dosage form of claim 18, wherein the corticosteroid is selected from the group consisting of beclomethasone, betamethasone, budesonide, dexamethasone, flunisolide, hydrocortisone, triamcinolone, fludrocortisone, fluocinolone, fluocinonide, fluticasone, methylprednisolone, mometasone, prednisone, and pharmacologically acceptable salts, esters, and derivatives thereof.

20. The dosage form of claim 19, wherein the corticosteroid is mometasone or a pharmacologically acceptable salt, ester, or a derivative thereof.

21. The dosage form of claim 16, wherein the active agent is a bronchodilator.

22. The dosage form of claim 21, wherein the bronchodilator is selected from the group consisting of albuterol, bitolterol, clenbuterol, fenoterol, formoterol, levalbuterol, metaproterenol, pirbuterol, procaterol, reproterol, rimiterol, salmeterol, terbutaline, and pharmacologically acceptable salts, esters, and derivatives thereof.

23. The dosage form of claim 1, further comprising a second active agent.

24. The dosage form of claim 23, wherein the first active agent is a corticosteroid and the second active agent is a bronchodilator.

25. The dosage form of claim 24, wherein the corticosteroid is mometasone furoate and the bronchodilator is selected from the group consisting of pirbuterol dihydrochloride, pirbuterol acetate, and levalbuterol sulfate.

26. The dosage form of claim 1, wherein the substantially moisture-impermeable outer surface effectively prohibits moisture from reaching the enclosed interior region when stored at 40° C. at 75% relative humidity for three days.

27. The dosage form of claim 1, further comprising a plasticizer.

28. The dosage form of claim 27, wherein the plasticizer is selected from the group consisting of glycerin, propylene glycol, polyethylene glycol, dibutyl sebacate, diethylphthalate, triethyl citrate, and combinations thereof.

29. A method for reducing moisture transmission in a dosage form, comprising: formulating at least one active agent into a capsule; and coating the capsule with a substantially moisture impermeable synthetic polymer to produce a dosage form having reduced moisture-transmission properties.

30. The method of claim 29, wherein the synthetic polymer is non-hydrolyzable.

31. The method of claim 29, wherein the capsule is comprised of hydroxypropyl methylcellulose or gelatin.

32. The method of claim 29, wherein the synthetic polymer is selected from the group consisting of polyesters, poly(nonhalogenated hydrocarbons), poly(halogenated hydrocarbons), poly(halogenated polyethers), polymers formed from dienes, poly(higher alkylene oxides), polyamides, polysiloxanes, polysilanes, poly(acrylonitriles), poly(lower alkylene oxides), acrylate polymers, polyacrylic acids and alternating copolymers, block copolymers, random copolymers, graft copolymers, terpolymers, block terpolymers, and random terpolymers thereof, and combinations of any of the foregoing.

33. The method of claim 33, wherein the synthetic polymer is selected from the group consisting of poly(ethylene terephthalate), poly(butylene terephthalate), poly(trimethylene terephthalate), poly(ethylene naphthalate), polyethylene, polypropylene, polystyrene, polyisobutylene, polymethylpentene, poly(tetrafluoro ethylene), poly(chlorotrifluoro ethylene), poly(vinyl chloride), poly(vinyl fluoride), poly(vinyl bromide), poly(vinyl iodide), poly(vinylidene chloride), poly(vinylidene fluoride), poly(vinylidene bromide), poly(vinylidene iodide), chlorinated polyether, polybutadiene, poly(dicyclopentadiene), poly(butylene oxide), poly(propylene oxide), poly(2,6-dimethylphenylene oxide), nylons, poly(dimethyl siloxane), poly(methylphenyl siloxane), poly(diphenyl siloxane), poly(methylphenyl silane), poly(acrylonitrile), poly(ethylene oxide), poly(methyl acrylate), poly(ethyl acrylate), polymethacrylic acid, polyethylacrylic acid, poly(methyl methacrylate), poly(ethyl methacrylate), and alternating copolymers, block copolymers, random copolymers, graft copolymers, terpolymers, block terpolymers, and random terpolymers thereof, and combinations of any of the foregoing.

34. The method of claim 33, wherein the synthetic polymer is selected from the group consisting of polyethylene, polypropylene, polystyrene, and combinations of any of the foregoing.

35. The method of claim 29, wherein the substantially moisture-impermeable synthetic material effectively prohibits moisture from reaching the enclosed interior when stored at 40° C. at 75% relative humidity for three days.

36. The method of claim 29, wherein the capsule further comprises a plasticizer.

37. The method of claim 36, wherein the plasticizer is selected from the group consisting of glycerin, propylene glycol, polyethylene glycol, dibutyl sebacate, diethylphthalate, triethyl citrate, and combinations thereof

38. The method of claim 29, further comprising the step of sterilizing the dosage form.

39. The method of claim 38, wherein the sterilization step is carried out by exposing the dosage form to radiation.

40. The method of claim 39, wherein the sterilization step is carried out by exposing the dosage form to ethylene oxide.

41. A method for reducing moisture transmission in a dosage form, comprising: formulating a capsule comprised of a substantially moisture impermeable synthetic polymer; and filling the capsule with an active agent, thereby providing a dosage form having reduced moisture-transmission properties.

42. The method of claim 41, wherein the synthetic polymer is non-hydrolyzable.

43. The method of claim 41, wherein the capsule further comprises hydroxypropyl methylcellulose or gelatin.

44. The method of claim 41, wherein the synthetic polymer is selected from the group consisting of polyesters, poly(nonhalogenated hydrocarbons), poly(halogenated hydrocarbons), poly(halogenated polyethers), polymers formed from dienes, poly(higher alkylene oxides), polyamides, polysiloxanes, polysilanes, poly(acrylonitriles), poly(lower alkylene oxides), acrylate polymers, polyacrylic acids and alternating copolymers, block copolymers, random copolymers, graft copolymers, terpolymers, block terpolymers, and random terpolymers thereof, and combinations of any of the foregoing.

45. The method of claim 44, wherein the synthetic polymer is selected from the group consisting of poly(ethylene terephthalate), poly(butylene terephthalate), poly(trimethylene terephthalate), poly(ethylene naphthalate), polyethylene, polypropylene, polystyrene, polyisobutylene, polymethylpentene, poly(tetrafluoro ethylene), poly(chlorotrifluoro ethylene), poly(vinyl chloride), poly(vinyl fluoride), poly(vinyl bromide), poly(vinyl iodide), poly(vinylidene chloride), poly(vinylidene fluoride), poly(vinylidene bromide), poly(vinylidene iodide), chlorinated polyether, polybutadiene, poly(dicyclopentadiene), poly(butylene oxide), poly(propylene oxide), poly(2,6-dimethylphenylene oxide), nylons, poly(dimethyl siloxane), poly(methylphenyl siloxane), poly(diphenyl siloxane), poly(methylphenyl silane), poly(acrylonitrile), poly(ethylene oxide), poly(methyl acrylate), poly(ethyl acrylate), polymethacrylic acid, polyethylacrylic acid, poly(methyl methacrylate), poly(ethyl methacrylate), and alternating copolymers, block copolymers, random copolymers, graft copolymers, terpolymers, block terpolymers, and random terpolymers thereof, and combinations of any of the foregoing.

46. The method of claim 45, wherein the synthetic polymer is selected from the group consisting of polyethylene, polypropylene, polystyrene, and combinations of any of the foregoing.

47. The method of claim 41, wherein the substantially moisture-impermeable synthetic material effectively prohibits moisture from reaching the enclosed interior when stored at 40° C. at 75% relative humidity for three days.

48. The method of claim 41, wherein the capsule further comprises a plasticizer.

49. The method of claim 48, wherein the plasticizer is selected from the group consisting of glycerin, propylene glycol, polyethylene glycol, dibutyl sebacate, diethylphthalate, triethyl citrate, and combinations thereof

50. The method of claim 41, further comprising the step of sterilizing the dosage form.

51. The method of claim 50, wherein the sterilization step is carried out by exposing the dosage form to radiation.

52. The method of claim 50, wherein the sterilization step is carried out by exposing the dosage form to ethylene oxide.

53. A method of treating a patient with an active agent comprising:
(a) providing a pharmaceutical dosage form comprised of a capsule having a substantially moisture-impermeable outer surface, an enclosed interior cavity, and an active agent;
(b) administering a therapeutically effective amount of the active agent to a patient in need thereof from the pharmaceutical dosage form.

54. The method of claim 53, wherein the active agent is selected from the group consisting of small drug molecules, polypeptides, proteins, and polynucleotides, and combinations thereof.

55. The method of claim 54, wherein the active agent is selected from the group consisting of narcotics, bronchodilators, corticosteroids, anti-cystic fibrosis agents, anti-diabetes agents, immunoregulatory agents, hormones, and hormone modulators.

56. The method of claim 55, wherein the active agent is selected from the group consisting of albuterol, bitolterol, clenbuterol, fenoterol, formoterol, levalbuterol, metaproterenol, pirbuterol, procaterol, reproterol, rimiterol, salmeterol, terbutaline, beclomethasone, betamethasone, budesonide, dexamethasone, flunisolide, hydrocortisone, triamcinolone, fludrocortisone, fluocinolone, fluocinonide, fluticasone, methylprednisolone, mometasone, prednisone, luteinizing hormone-releasing hormone, nafarelin, goserelin, leuprolide, cyclosporin, calcitonin, granulocyte colony-stimulating factor, growth hormones, heparin, parathyroid hormone, insulin, interferon, α₁-proteinase inhibitor, interleukins, cystic fibrosis transmembrane conductance gene, α₁-antitrypsin gene, and pharmacologically acceptable salts, esters, and derivatives thereof.

57. The method of claim 53, wherein the patient is suffering from asthma, exercise-induced asthma, bronchitis, bronchospasm, rhinitis or emphysema.

58. The method of claim 53, wherein the patient is suffering from cystic fibrosis.

59. The method of claim 53, wherein the patient is suffering from diabetes.

60. The method of claim 53, wherein the active agent is administered by pulmonary inhalation.

61. The method of claim 60, wherein the patient uses a dry powder inhaler to administer the active agent.
